(19) United States
(12) Patent Application Publication
(10) Pub. No.: US 2018/0009601 A1
(43) Pub. Date: Jan. 11, 2018

(54) Preparing Beverages Containing Cannabinoids Using Containers with Polymer Matrices

(71) Applicant: Zen Potion, Inc., Pleasanton, CA (US)

(72) Inventor: Seung Young BYUN, Pleasanton, CA (US)

(21) Appl. No.: 15/710,682

(22) Filed: Sep. 20, 2017

Related U.S. Application Data
(63) Continuation of application No. PCT/US2016/023211, filed on Mar. 18, 2016.
(60) Provisional application No. 62/136,379, filed on Mar. 20, 2015.
(60) Provisional application No. 62/253,618, filed on Nov. 10, 2015.

(51) Int. Cl. B65D 85/808; A61K 31/05; A61K 31/352

(52) U.S. Cl. CPC B65D 85/808; A23L 33/00; A61K 31/352; A61K 31/05

(57) ABSTRACT

A liquid-permeable container includes an enclosure that includes a liquid-permeable membrane. The liquid-permeable membrane permits liquid to enter into and exit from the enclosure at least through the liquid-permeable membrane. The liquid-permeable container also includes one or more polymer matrices that include one or more cannabinoid compounds. A package includes the liquid-permeable container and a water-resistant package enveloping the liquid-permeable container. Methods for manufacturing the liquid-permeable container and methods for preparing a beverage containing cannabinoids using the liquid-permeable container are also described.

### RELATED APPLICATIONS

This application is a continuation application of PCT/US2016/023211, filed Mar. 18, 2016, which claims priority to U.S. Provisional Patent Application 62/136,379, filed Mar. 20, 2015 and U.S. Provisional Patent Application 62/253,618, filed Nov. 10, 2015. All of these applications are incorporated by reference herein in their entireties.

### TECHNICAL FIELD

This application relates to the field of containers, and particularly to containers with water soluble cannabinoids.

### BACKGROUND

Cannabinoids generally refer to chemical compounds that act on cannabinoid receptors on cells. Cannabinoids include cannabinoids derived from cannabis, such as tetrahydrocannabinol, cannabidiol, cannabinol, cannabigerol, tetrahydrocannabivarin, cannabidivarin, and cannabichromene. Cannabinoids also include cannabinoids derived from other plants. Cannabinoids further include synthetic cannabinoids.

Cannabinoids are often used for medical purposes. Cannabinoids can be administered by various methods, such as oral ingestion, smoking, vaporizing, etc.

### SUMMARY

Cannabinoids are generally insoluble in water, and thus, are often extracted and stored in oil-based media (e.g., oil solution or oil-containing powder). Because hydrophobicity of oil-based media and insolubility of cannabinoids hinder interaction of the cannabinoids in oil-based media with aqueous solutions, it was believed that cannabinoids in an oil-based medium would retain their potency even if the oil-based medium is mixed with an aqueous solution.

The inventors of this application have found that, surprisingly, when cannabinoids are mixed with aqueous solution, the potency of the cannabinoids decreases significantly over time. Thus, beverages (e.g., aqueous beverage) containing cannabinoids cannot be stored for long without losing the potency of cannabinoids.

A number of embodiments that overcome the limitations and disadvantages described above are presented in more detail below. These embodiments provide containers and methods described herein. By storing cannabinoid compounds in polymer matrices in a sealed container and mixing the cannabinoid compounds with a beverage (e.g., water) only within a certain time before consumption, the loss of potency is reduced. Such containers provide additional benefits, such as storing cannabinoid compounds safely in a sealed environment so that the cannabinoid compounds are not accidentally released. In addition, the containers described herein provide a simplified mechanism for mixing beverages and cannabinoid compounds, thereby facilitating preparation of beverages containing cannabinoids.

The inventors of this application have also found that cannabinoid compounds do not mix well with an aqueous beverage due to the hydrophobicity of the oil-based media and insolubility of the cannabinoid compounds in water. Thus, releasing cannabinoid compounds into an aqueous beverage leads to a non-homogeneous mixture of cannabinoid compounds and the aqueous beverage (e.g., the oil-based media including cannabinoid compounds floating on top of the aqueous beverage), which results in a non-uniform rate of ingesting cannabinoid compounds while drinking the aqueous beverage and reduced consumer satisfaction. As described below, the cannabinoid compounds included in one or more polymer matrices are used to address this problem. For example, the one or more polymer matrices are used to improve the solubility of the cannabinoid compounds or at least create a more homogeneous mixture of cannabinoid compounds and the aqueous beverage. The one or more polymer matrices also facilitate maintaining the potency of cannabinoid compounds while stored separately from the aqueous beverage.

In accordance with some embodiments, a liquid-permeable container includes an enclosure that includes a liquid-permeable membrane. The liquid-permeable membrane permits liquid to enter into and exit from the enclosure at least through the liquid-permeable membrane. The liquid-permeable container also includes one or more polymer matrices that include one or more cannabinoid compounds.

In some embodiments, the one or more polymer matrices include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, the water-permeable membrane includes a nonwoven fabric. In some embodiments, the entire enclosure is made with the nonwoven fabric.

In some embodiments, the one or more polymer matrices are located within the enclosure. In some embodiments, the one or more polymer matrices are applied on the water-permeable membrane.

In some embodiments, the one or more cannabinoid compounds include one or more of: tetrahydrocannabinolic acid, tetrahydrocannabinol, and cannabidiol. In some embodiments, the one or more cannabinoid compounds include tetrahydrocannabinol. In some embodiments, the one or more cannabinoid compounds include cannabidiol. In some embodiments, the one or more cannabinoid compounds include tetrahydrocannabinolic acid.

In some embodiments, the water-permeable container is configured to retain the one or more polymer matrices prior to the enclosure being exposed to the liquid.

In some embodiments, the enclosure is a tea bag.

In some embodiments, the liquid-permeable container includes one or more flavoring substances.

In some embodiments, the liquid is water.

In some embodiments, a potency of the one or more cannabinoid compounds decreases over time subsequent to the one or more cannabinoid compounds are exposed to the liquid.

In accordance with some embodiments, a method of manufacturing a liquid-permeable container includes obtaining an enclosure that includes a liquid-permeable membrane. The liquid-permeable membrane permits liquid to enter into and exit from the enclosure through the liquid-permeable membrane. The method includes providing the one or more polymer matrices, that include one or more cannabinoid compounds; and sealing the enclosure.

In accordance with some embodiments, a method for preparing a beverage containing cannabinoids includes obtaining a liquid-permeable container; and exposing the liquid-permeable container to liquid, thereby allowing the liquid to enter into and exit from the enclosure at least through a liquid-permeable membrane and releasing one or more cannabinoid compounds to the liquid.

In accordance with some embodiments, a package includes a liquid-permeable container and a water-resistant package enveloping the liquid-permeable container.

In accordance with some embodiments, a container for preparing a beverage includes an enclosure that includes one or more side walls, a pierceable base sealingly coupled with the one or more side walls, and a pierceable lid sealingly coupled with the one or more side walls opposite to the base. The container also includes one or more polymer matrices, that include one or more cannabinoid compounds, located within the enclosure.

In some embodiments, the container includes a filter.

In some embodiments, the one or more polymer matrices are located between the filter and the pierceable base.

In some embodiments, the pierceable lid includes a metal sheet.

In some embodiments, the pierceable base includes a metal sheet. In some embodiments, the pierceable base includes plastic.

In some embodiments, the one or more side walls and the pierceable base include a same material.

In some embodiments, the one or more side walls and the pierceable base have a same material composition.

In some embodiments, the enclosure prevents liquid to enter into and exit from the enclosure prior to piercing of the pierceable base and/or the pierceable lid.

In some embodiments, the container includes coffee powders.

In some embodiments, the container includes a mixture of a first set of food items with the one or more polymer matrices thereon and a second set of food items without the one or more polymer matrices thereon.

In accordance with some embodiments, a method of manufacturing a container includes obtaining one or more side walls of an enclosure, sealingly coupled with a pierceable base; providing one or more polymer matrices that include one or more cannabinoid compounds; and sealing the one or more side walls with a pierceable lid opposite to the pierceable base so that the one or more polymer matrices are sealingly contained within the enclosure.

In accordance with some embodiments, a method for preparing a beverage containing cannabinoids includes obtaining a container; piercing a pierceable base of the container; piercing a pierceable lid of the container; and providing liquid so that the liquid enters into an enclosure of the container through the piercing in the pierceable lid and exits from the enclosure of the container through the piercing in the pierceable base of the container, thereby forming a beverage containing one or more cannabinoid compounds.

In accordance with some embodiments, a water-resistant container includes one or more polymer matrices, that include one or more cannabinoid compounds; and a water-resistant enclosure enclosing the one or more polymer matrices.

In some embodiments, the water-resistance enclosure is tearable.

In some embodiments, the water-resistant enclosure includes a metal sheet.

In some embodiments, the water-resistant enclosure includes plastic.

In some embodiments, the water-resistant enclosure prevents release of the one or more polymer matrices from the water-resistant enclosure prior to tearing of the water-resistant enclosure.

In some embodiments, a potency of the one or more cannabinoid compounds decreases over time subsequent to the one or more cannabinoid compounds are exposed to water.

In some embodiments, the water-resistant container includes one or more flavoring substances.

In some embodiments, the water-resistant container includes a mixture of a first set of food items with the one or more polymer matrices thereon and a second set of food items without the one or more polymer matrices thereon.

In accordance with some embodiments, a method of manufacturing a water-resistant container includes obtaining a water-resistant enclosure; providing the one or more polymer matrices that include one or more cannabinoid compounds; and sealing the water-resistant enclosure so that the one or more polymer matrices are sealingly contained within the water-resistant enclosure.

In accordance with some embodiments, a method for preparing a beverage containing cannabinoids includes obtaining a water-resistant container; tearing the water-resistant enclosure of the water-resistant container; and releasing the one or more cannabinoid compounds from the water-resistant enclosure into liquid, thereby forming a beverage containing the one or more cannabinoid compounds.

In accordance with some embodiments, a method for preparing a food item includes obtaining a plurality of food items; and applying to the plurality of food items a coating solution that includes a solvent, one or more polymers, and one or more cannabinoid compounds. The method also includes evaporating the solvent so that one or more polymer matrices that include the one or more polymers and the one or more cannabinoid compounds are formed on surfaces of the plurality of food items.

In accordance with some embodiments, a food item includes one or more polymer matrices on a surface of the food item. The one or more polymer matrices include one or more cannabinoid compounds.

In accordance with some embodiments, a mixture of food items includes a first set of a plurality of food items with one or more polymer matrices thereon; and a second set of a plurality of food items without the one or more polymer matrices thereon.

In accordance with some embodiments, a beverage stirrer includes a rod; and a container coupled to a first end of the rod. The container releasably stores one or more cannabinoid compounds. In some embodiments, the container releasably stores one or more polymer matrices that include one or more cannabinoid compounds.

In some embodiments, the container includes a liquid-permeable membrane, wherein the liquid-permeable membrane permits liquid to enter into and exit from the container at least through the liquid-permeable membrane.

In some embodiments, the container includes a water soluble membrane that is configured to prevent egress of the one or more cannabinoid compounds until at least a portion of the water soluble membrane is dissolved.

In accordance with some embodiments, a method for preparing a beverage containing cannabinoids includes obtaining a beverage stirrer and exposing the beverage stirrer to liquid, thereby releasing one or more cannabinoid compounds to the liquid.

In accordance with some embodiments, a method includes obtaining a beverage stirrer that comprises a rod and a container coupled to a first end of the rod; placing in the container one or more cannabinoid compounds; and sealing the container. In some embodiments, sealing the container includes sealing the container with a liquid-permeable membrane. In some embodiments, sealing the container includes sealing the container with a water-soluble membrane.

In accordance with some embodiments, a package includes a beverage stirrer and a water-resistant package enveloping the beverage stirrer.

### BRIEF DESCRIPTION OF THE DRAWINGS

For a better understanding of the aforementioned aspects as well as additional aspects and embodiments thereof, reference should be made to the Description of Embodiments below, in conjunction with the following drawings.

FIG. 1A is a skeletal formula representing a chemical structure of tetrahydrocannabinolic acid.

FIG. 1B is a skeletal formula representing a chemical structure of tetrahydrocannabinol.

FIG. 1C is a skeletal formula representing a chemical structure of cannabidiol.

FIGS. 2A-2B are perspective views of a liquid-permeable container in accordance with some embodiments.

FIG. 2C is a perspective view of a liquid-permeable container in accordance with some embodiments.

FIG. 2D is a perspective view of a package including a liquid-permeable container in accordance with some embodiments.

FIG. 3 is an exploded view of a container for preparing a beverage in accordance with some embodiments.

FIGS. 4A-4B are perspective views of a water-resistant container in accordance with some embodiments.

FIGS. 5A-5D illustrate a method for preparing a food item in accordance with some embodiments.

FIG. 6 illustrates a method for preparing a beverage containing cannabinoids in accordance with some embodiments.

FIG. 7 illustrates a method for manufacturing a liquid-permeable container in accordance with some embodiments.

FIG. 8 illustrates a method for preparing a beverage containing cannabinoids in accordance with some embodiments.

FIG. 9 illustrates a method for manufacturing a container in accordance with some embodiments.

FIG. 10 illustrates a method for preparing a beverage containing cannabinoids in accordance with some embodiments.

FIG. 11 illustrates a method for manufacturing a water-resistant container in accordance with some embodiments.

FIG. 12 illustrates a method for preparing a food item in accordance with some embodiments.

FIG. 13 illustrates a beverage stirrer in accordance with some embodiments.

Like reference numerals refer to corresponding parts throughout the drawings. Drawings are not necessarily drawn to scale unless explicitly stated otherwise.

### DESCRIPTION OF EMBODIMENTS

Containers with cannabinoids in polymer matrices, and food items with cannabinoids in polymer matrices, and methods for making and using them are described.

Reference will now be made to certain embodiments, examples of which are illustrated in the accompanying drawings. While the embodiments are described in conjunction with the drawings, it will be understood that it is not intended to limit the scope of the claims to these particular embodiments alone. In the following detailed description, numerous specific details are set forth in order to provide a thorough understanding of the subject matter presented herein. But it will be apparent to one skilled in the art that the subject matter may be practiced without these specific details. In other instances, well-known methods, procedures, and components have not been described in detail so as not to unnecessarily obscure certain aspects of the embodiments.

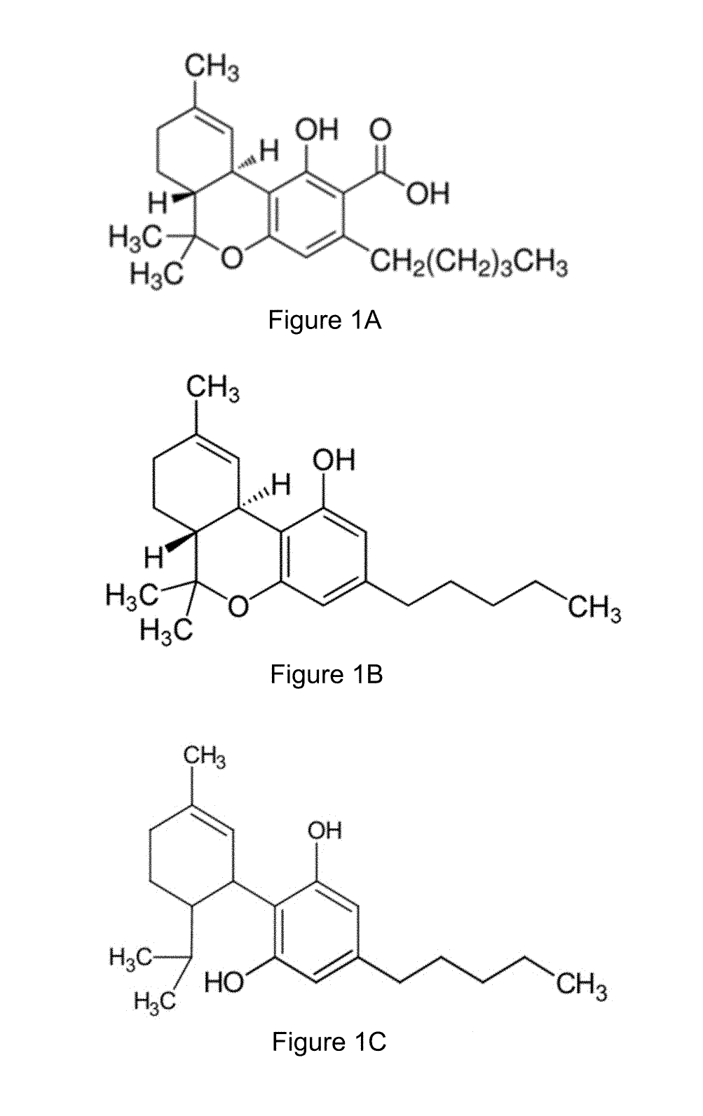

FIG. 1A is a skeletal formula representing a chemical structure of tetrahydrocannabinolic acid. In some embodiments, tetrahydrocannabinolic acid refers to (6aR,10aR)-1-hydroxy-6,6, 9-trimethyl-3-pentyl-6a,7,8,10a-tetrahydro-6h-benzo[c]chromene-2-carboxylic acid.

FIG. 1B is a skeletal formula representing a chemical structure of tetrahydrocannabinol. In some embodiments, the term tetrahydrocannabinol refers to (−)-(6aR,10aR)-6,6,9-Trimethyl-3-pentyl-6a,7,8,10a-tetrahydro-6H-benzo[c]chromen-1-ol. In some embodiments, tetrahydrocannabinol is also called (−)-trans-Δ⁹-tetrahydrocannabinol.

FIG. 1C is a skeletal formula representing a chemical structure of cannabidiol. In some embodiments, cannabidiol refers to 2-[(1R,6R)-6-isopropenyl-3-methylcyclohex-2-en-1-yl]-5-pentylbenzene-1,3-diol.

Although FIGS. 1A-1C illustrate particular skeletal formulas of the exemplary chemicals, a person having ordinary skill in the art would understand that a same chemical may be represented using different skeletal formulas. For example, the CH₂(CH₂)₃CH₃ chain shown in FIG. 1A may be represented using multiple connections of carbon atoms (e.g., CH₂), as represented in FIGS. 1B and 1C. Optionally, stereochemistry may be also indicated in skeletal formulas, as shown in FIGS. 1A and 1B.

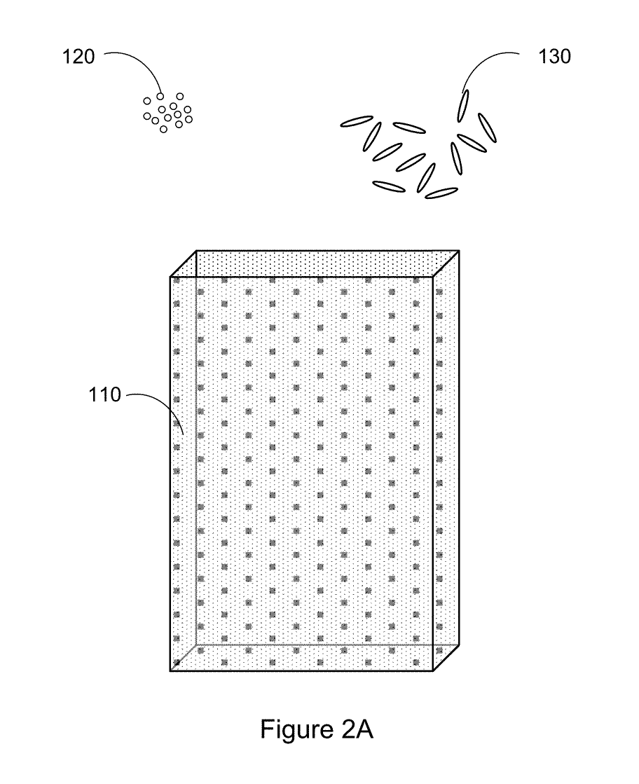

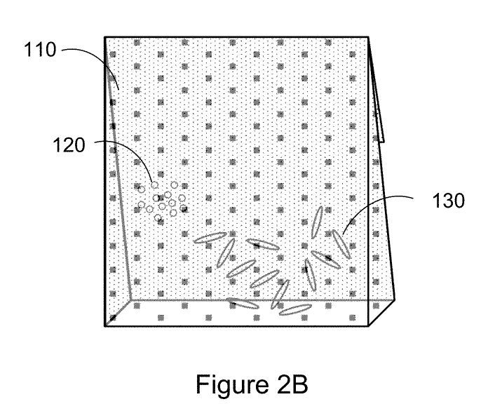

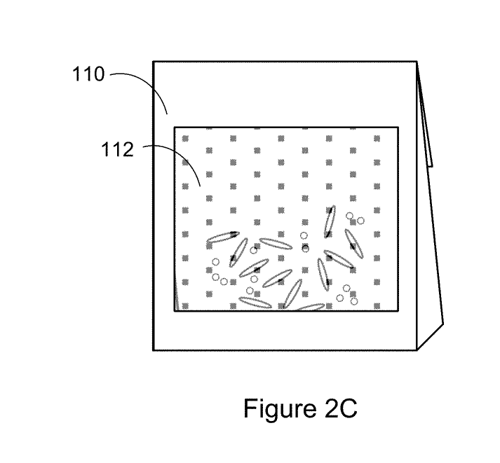

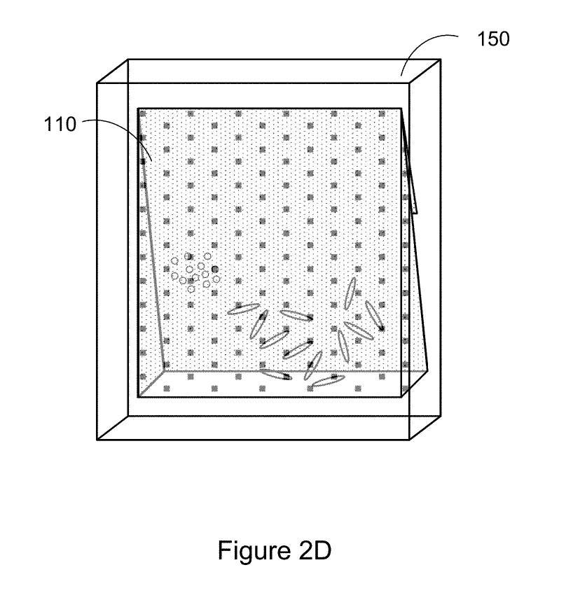

FIGS. 2A-2B are perspective views of a liquid-permeable container in accordance with some embodiments.

FIG. 2A illustrates the liquid-permeable container. In FIG. 2A, the liquid-permeable container includes an enclosure 110. In some embodiments, the entire enclosure 110 is made of a liquid-permeable membrane, as shown in FIG. 2A. The liquid-permeable membrane permits liquid (e.g., water) to enter into and exit from the enclosure 110 at least through the liquid-permeable membrane (e.g., the liquid-permeable membrane is a water-permeable membrane). For example, a tea bag allows water to enter into and exit from the tea bag through the tea bag fabric.

In some embodiments, the liquid-permeable membrane includes a fabric. In some embodiments, the liquid-permeable membrane includes a nonwoven fabric. In some embodiments, the liquid-permeable membrane includes one or more of filter paper, silk, or food grade plastic (e.g., nylon, polyactide, polypropylene, and polyvinyl chloride). In some embodiments, the liquid-permeable membrane is made of one or more of filter paper, silk, or food grade plastic.

In some embodiments, the enclosure 110 has a shape of a bag (e.g., a tea bag). In some embodiments, the enclosure 110 is a rectangular bag, as shown in FIG. 2A. In some embodiments, the enclosure 110 is a circular bag. In some embodiments, the enclosure 110 is a tetrahedral bag.

FIG. 2A also illustrates one or more polymer matrices 120. The one or more polymer matrices include one or more cannabinoid compounds (e.g., tetrahydrocannabinolic acid, tetrahydrocannabinol, and/or cannabidiol illustrated in FIGS. 1A-1C). In some embodiments, the one or more cannabinoid compounds include one or more of: tetrahydrocannabinolic acid, tetrahydrocannabinol, and cannabidiol. In some embodiments, the one or more cannabinoid compounds include tetrahydrocannabinol. In some embodiments, the one or more cannabinoid compounds include cannabidiol. In some embodiments, the one or more cannabinoid compounds include tetrahydrocannabinolic acid.

In some embodiments, the one or more polymer matrices 120 also include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, a polymer matrix of the one or more polymer matrices 120 includes at least 20 wt. % of polyoxazoline. In some embodiments, the polymer matrix of the one or more polymer matrices includes at least 50 wt. % of polyoxazoline. In some embodiments, the polyoxazoline has a molar mass of at least 40,000 g/mol. In some embodiments, the polyoxazoline has a molar mass of at least 80,000 g/mol. In some embodiments, the polyoxazoline has a molar mass of at least 150,000 g/mol. In some embodiments, the polyoxazoline has a molar mass of at least 200,000 g/mol. In some embodiments, the polymer matrix of the one or more polymer matrices includes at least 0.001% by weight of cannabinoid compounds. In some embodiments, the polymer matrix of the one or more polymer matrices includes at least 0.1% by weight of cannabinoid compounds. In some embodiments, the polymer matrix of the one or more polymer matrices includes 0.5-30% by weight of cannabinoid compounds. In some embodiments, the polymer matrix of the one or more polymer matrices includes at least 70% by weight of cannabinoid compounds. In some embodiments, the polyoxazoline is a homopolymer of 2-alkyl-oxazoline. In some embodiments, the 2-alkyl-oxazoline is selected from 2-methyl-oxazoline, 2-ethyl-oxazoline, 2-propyl-oxazoline, 2-butyl-oxazoline and any combination thereof.

FIG. 2A illustrates that, in some embodiments, one or more flavoring substances 130 (e.g., tea leaves, tea extracts, coffee powder, soda extracts, sugar, etc.) are also provided. In some embodiments, the liquid-permeable container includes tea leaves. In some embodiments, the liquid-permeable container includes tea extracts.

FIG. 2B illustrates that the one or more polymer matrices 120 and optionally the one or more flavoring substances 130 are placed inside the enclosure 110. FIG. 2B also illustrates that the enclosure 110 is closed to prevent the one or more polymer matrices 120 from getting released from the enclosure 110. In some embodiments, the liquid-permeable container is configured to retain the one or more polymer matrices prior to the enclosure 110 being exposed to the liquid. For example, the liquid-permeable membrane allows liquid to permeate but prevents the one or more polymer matrices 120 (e.g., in a powder form) from going through the liquid-permeable membrane. In some embodiments, the liquid-permeable membrane has a mesh, and a size of the mesh is selected to allow liquid to permeate but prevent the one or more polymer matrices 120 (e.g., in a powder form) from going through the liquid-permeable membrane.

In some embodiments, the one or more polymer matrices 120 are located within the enclosure 110, as shown in FIG. 2B.

In some embodiments, the one or more polymer matrices 120 are applied on the liquid-permeable membrane. For example, instead of placing the one or more polymer matrices 120 (e.g., in a powder form) inside the enclosure 110, a film or films of the one or more polymer matrices may be formed on the liquid-permeable membrane. In some embodiments, the film of the one or more polymer matrices are formed on an inside surface of the liquid-permeable membrane to prevent accidental release of the one or more polymer matrices (e.g., before the enclosure 110 is exposed to liquid).

In some embodiments, the liquid-permeable container includes a mixture of a first set of food items with the one or more polymer matrices thereon (e.g., coffee powder with one or more polymer matrices) and a second set of food items without the one or more polymer matrices thereon (e.g., coffee powder, or sugar powder, without one or more polymer matrices). In some embodiments, the liquid-permeable container includes one or more other food items.

In some embodiments, a potency of the one or more cannabinoid compounds decreases over time subsequent to the one or more cannabinoid compounds are exposed to the liquid. Thus, by storing the one or more cannabinoid compounds in the one or more polymer matrices 120, separate from the liquid, the potency of the one or more cannabinoid compounds is maintained for an extended period of time. In addition, the one or more polymer matrices 120 improves the solubility of the one or more cannabinoid compounds in aqueous solution (e.g., water), thereby facilitating formation of a uniform mixture of the one or more cannabinoid compounds when the one or more cannabinoid compounds are exposed to the aqueous solution.

FIG. 2C is a perspective view of a liquid-permeable container in accordance with some embodiments. In FIG. 2C, the enclosure 110 includes a liquid-permeable membrane 112. As shown in FIG. 2C, in some embodiments, the liquid-permeable membrane 112 encompasses less than the entire surface of the enclosure 110 (e.g., the liquid-permeable membrane 112 is a window in the enclosure 110).

FIG. 2D is a perspective view of a package 150 including a liquid-permeable container in accordance with some embodiments. FIG. 2D illustrates that the enclosure 110 of the liquid-permeable container is enveloped by a water-resistant package 150 (e.g., plastic bag). The water-resistant package 150 prevents the enclosure 110 from getting exposed to liquid prior to use (e.g., until the enclosure 110 is taken out of the water-resistant package 150 by a user), thereby prolonging the potency of the one or more cannabinoid compounds in the one or more polymer matrices.

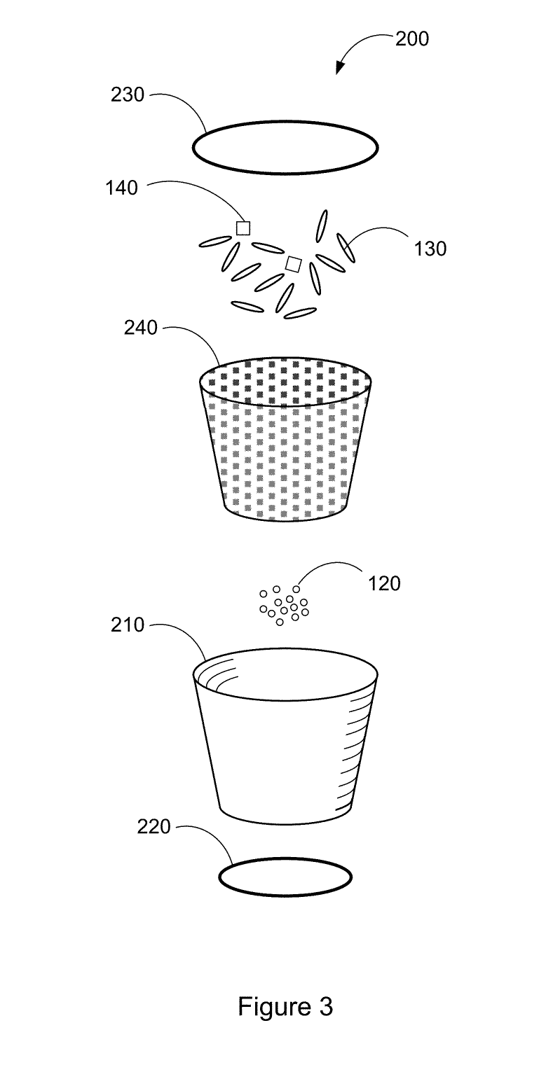

FIG. 3 is an exploded view of a container 200 for preparing a beverage in accordance with some embodiments. In some embodiments, the container 200 is a single serving container. In some embodiments, the container 200 is configured for use in a single serving beverage apparatus (e.g., a single serving brewing apparatus).

The container 200 includes an enclosure that includes one or more side walls 210, a pierceable base 220 sealingly coupled with the one or more side walls 210, and a pierceable lid 230 sealingly coupled with the one or more side walls 210 opposite to the base 220. In some embodiments, the one or more side walls 210 and the pierceable base 220 are integrally formed (e.g., in a single molding or stamping process).

In some embodiments, the one or more side walls 210 and the pierceable base 220 include a same material. In some embodiments, the one or more side walls 210 and the pierceable base 220 have a same material composition (e.g., a layer of a metal sheet covered by a plastic layer).

In some embodiments, the pierceable base 220 includes a metal sheet (e.g., aluminum). In some embodiments, the pierceable base 220 includes plastic.

In some embodiments, the pierceable lid 230 includes a metal sheet (e.g., aluminum).

In some embodiments, the enclosure prevents liquid to enter into and exit from the enclosure prior to piercing of the pierceable base and/or the pierceable lid. Piercing the pierceable base forms one or more holes in the pierceable base through which liquid is allowed to enter into and exit from the enclosure. Piercing the pierceable lid forms one or more holes in the pierceable lid through which liquid is allowed to enter into and exit from the enclosure. In some embodiments, both the pierceable base and the pierceable lid are pierced to facilitate the liquid to enter into and exit from the enclosure (e.g., the liquid may enter into the enclosure through the one or more holes in the pierceable lid 230 and exit from the enclosure through the one or more holes in the pierceable base 220). In some embodiments, the liquid enters into the enclosure through the one or more holes in the pierceable base 220 and exits from the enclosure through the one or more holes in the pierceable lid 230.

The container 200 also includes one or more polymer matrices 120 that include one or more cannabinoid compounds, located within the enclosure.

In some embodiments, the one or more polymer matrices 120 include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, the container includes a filter 240. In some embodiments, the container includes the filter 240 and coffee grinds so that remaining coffee grinds are filtered by the filter 240. In some embodiments, the container includes the filter 240 and tea leaves so that remaining tea leaves are filtered by the filter 240.

In some embodiments, the container does not include a filter 240. For powder that completely dissolves (e.g., cocoa powder, tea extracts, soda extracts, etc.), a filter may not be necessary. In some embodiments, the container includes cocoa powder.

In some embodiments, the one or more polymer matrices 120 are located between the filter 240 and the pierceable base 220. In some cases, placing the one or more polymer matrices 120 between the filter 240 and the pierceable base 220 facilitates dispensing of the one or more cannabinoid compounds from the container 200. In some embodiments, coffee powder (or any other flavoring substances) is located between the filter 240 and the pierceable lid 230. In some embodiments, tea leaves are located between the filter 240 and the pierceable lid 230.

In some embodiments, the one or more cannabinoid compounds include one or more of: tetrahydrocannabinolic acid, tetrahydrocannabinol, and cannabidiol. In some embodiments, the one or more cannabinoid compounds include tetrahydrocannabinol. In some embodiments, the one or more cannabinoid compounds include cannabidiol. In some embodiments, the one or more cannabinoid compounds include tetrahydrocannabinolic acid.

In some embodiments, a potency of the one or more cannabinoid compounds decreases over time subsequent to the one or more cannabinoid compounds are exposed to water.

In some embodiments, the container 200 includes one or more flavoring substances as shown in FIG. 3. In FIG. 3, the container 200 includes a first set of flavoring substances 130 (e.g., coffee powder, soda extracts, tea extracts, tea leaves, etc.). In some embodiments, the container 200 also includes a second set of flavoring substances 140 (e.g., coffee powder, soda extracts, tea extracts, tea leaves, etc.). In some embodiments, the second set of flavoring substances 140 is distinct from the first set of flavoring substances 130. In some embodiments, the container 200 includes one or more additional flavoring substances.

In some embodiments, the container 200 includes coffee powders. In some embodiments, the container 200 includes cocoa powder. In some embodiments, the container 200 includes tea extracts. In some embodiments, the container 200 includes soda extracts (e.g., extracts for a cold soda).

In some embodiments, the container includes a mixture of a first set of food items with the one or more polymer matrices thereon (e.g., coffee powder coated with one or more polymer matrices) and a second set of food items without the one or more polymer matrices thereon (e.g., coffee powder, or sugar powder, without a coating of one or more polymer matrices). In some embodiments, the liquid-permeable container includes one or more other food items.

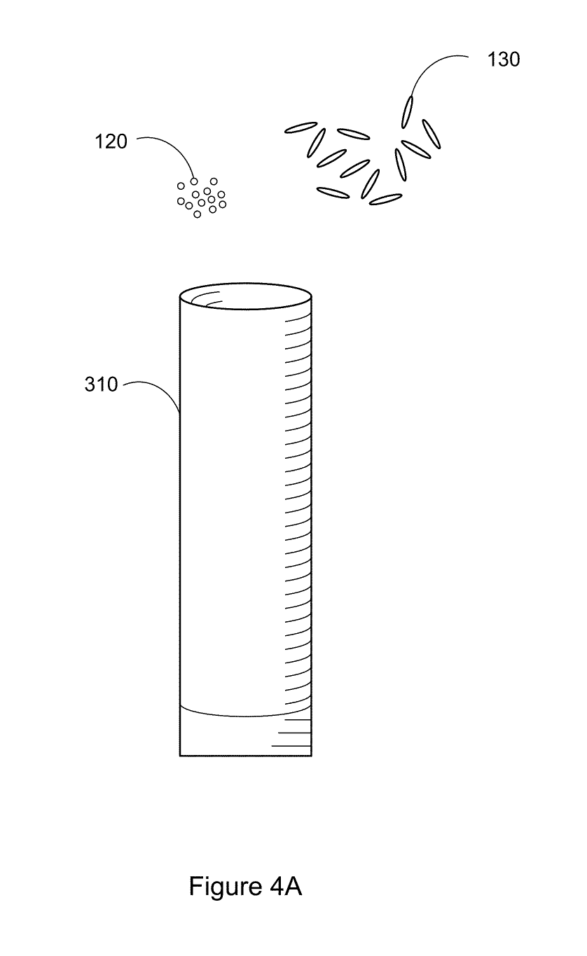

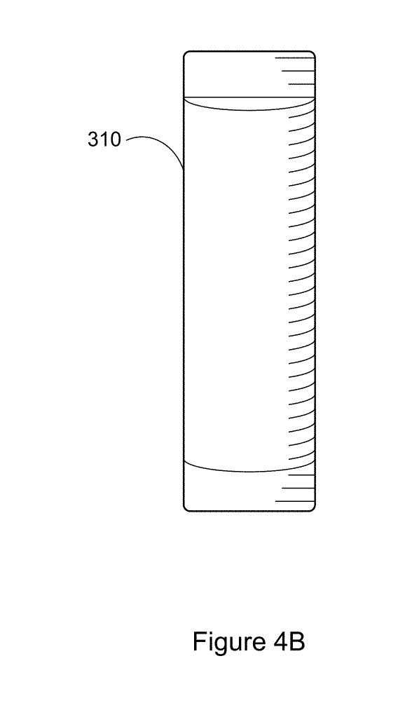

FIGS. 4A-4B are perspective views of a water-resistant container in accordance with some embodiments. In some embodiments, the water-resistant container is a stick pack.

FIG. 4A illustrates one or more polymer matrices 120 that include one or more cannabinoid compounds.

In some embodiments, the one or more polymer matrices 120 include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, the one or more cannabinoid compounds include one or more of: tetrahydrocannabinolic acid, tetrahydrocannabinol, and cannabidiol. In some embodiments, the one or more cannabinoid compounds include tetrahydrocannabinol. In some embodiments, the one or more cannabinoid compounds include cannabidiol. In some embodiments, the one or more cannabinoid compounds include tetrahydrocannabinolic acid.

FIG. 4A also illustrates a water-resistant enclosure 310. The one or more polymer matrices 120 are placed inside the water-resistant enclosure 310 so that the water-resistant enclosure encloses the one or more polymer matrices 120.

In some embodiments, the water-resistance enclosure is tearable. For example, the water-resistance enclosure may be made of a material that is configured to tear open.

In some embodiments, the water-resistant enclosure includes a metal sheet (e.g., aluminum foil). In some embodiments, the water-resistant enclosure includes plastic (e.g., a plastic coating). In some embodiments, the water-resistant enclosure includes a layer of a metal sheet and a layer of plastic or plastic coated paper.

Also shown in FIG. 4A are one or more flavoring substances 130. In some embodiments, the water-resistant container includes one or more flavoring substances 130.

In some embodiments, the water-resistant container includes coffee powders. In some embodiments, the water-resistant container includes cocoa powder (e.g., a mixture of cocoa powder, sugar, and milk powder). In some embodiments, the water-resistant container includes soda extracts.

In some embodiments, the water-resistant container includes a mixture of a first set of food items with the one or more polymer matrices thereon (e.g., coffee powder with one or more polymer matrices) and a second set of food items without the one or more polymer matrices thereon (e.g., coffee powder, or sugar powder, without one or more polymer matrices). In some embodiments, the liquid-permeable container includes one or more other food items.

FIG. 4B illustrates that the one or more polymer matrices 120 (shown in FIG. 4A) and optionally the one or more flavoring substances 130 (shown in FIG. 4A) are placed in the water-resistant enclosure 310 and the water-resistant enclosure 310 is sealed.

In some embodiments, the water-resistant enclosure prevents release of the one or more polymer matrices from the water-resistant enclosure prior to tearing of the water-resistant enclosure.

In some embodiments, a potency of the one or more cannabinoid compounds decreases over time subsequent to the one or more cannabinoid compounds are exposed to water.

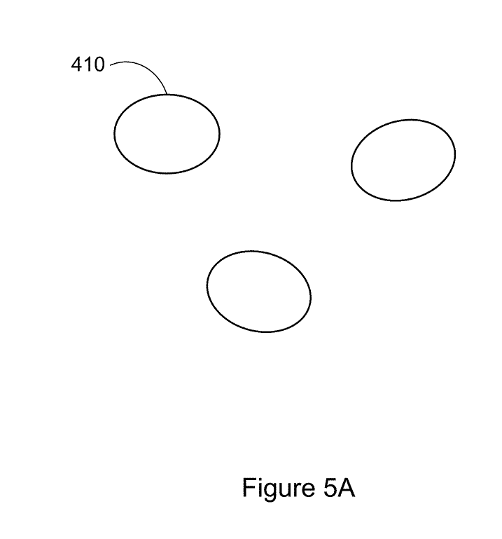

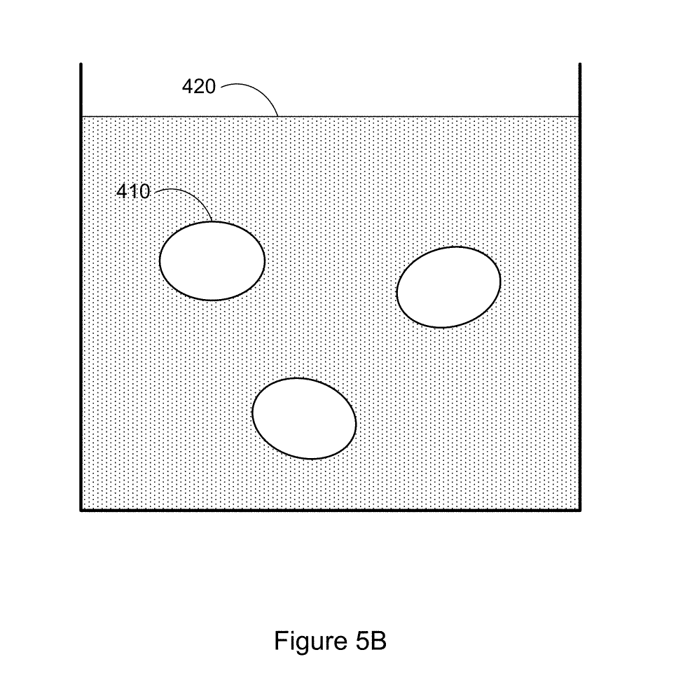

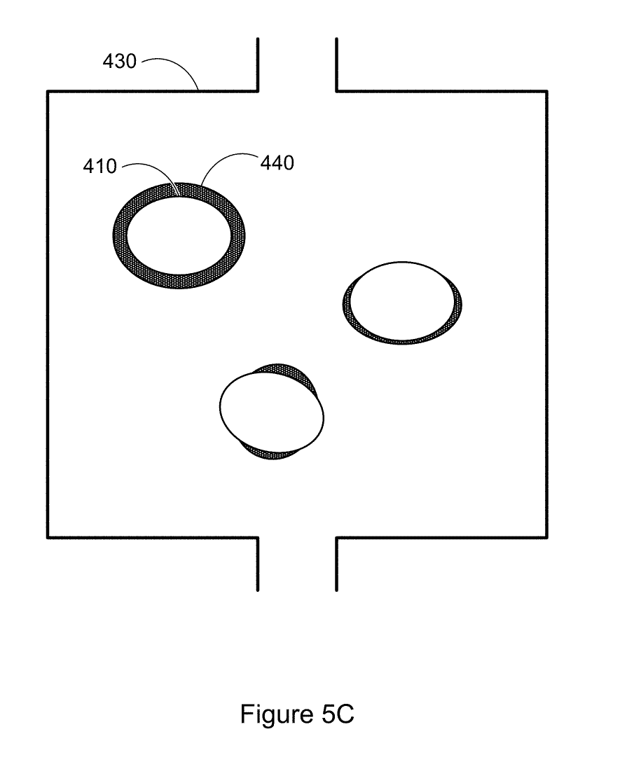

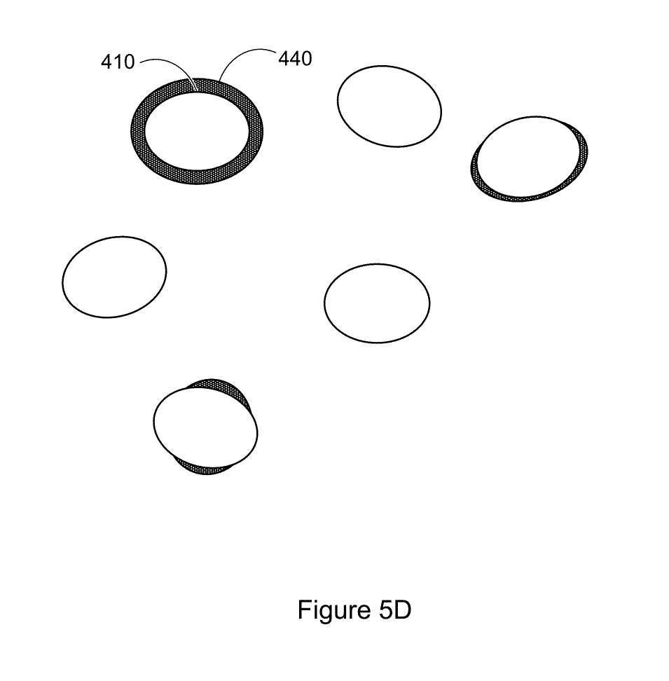

FIGS. 5A-5D illustrate a method for preparing a food item in accordance with some embodiments.

FIG. 5A shows a plurality of food items 410 (e.g., coffee grinds, coffee powder, coffee beans, sugar powder, tea leaves, tea balls, etc.). As used herein, a tea ball refers to a collection of tea leaves. In some embodiments, a tea ball includes a bundle of dried tea leaves wrapped around a dried flower. In some embodiments, a tea ball has a shape of a sphere or a spheroid.

FIG. 5B illustrates that a cannabinoid solution 420 is applied to the plurality of food items 410. In some embodiments, the cannabinoid solution 420 includes a solvent, one or more polymers, and one or more cannabinoid compounds.

FIG. 5C illustrates that the plurality of food items 410 with the cannabinoid solution 420 applied thereon is dried so that the solvent in the cannabinoid solution is evaporated, thereby leaving one or more polymer matrices 440 on the plurality of food items 410. In some embodiments, evaporating the solvent forms a coating of one or more polymer matrices on one or more food items (e.g., a food item is encapsulated by the coating of the one or more polymer matrices). In some embodiments, evaporating the solvent forms a film of one or more polymer matrices on one or more food items (e.g., the one or more polymer matrices form a continuous film or contiguous islands of the one or more polymer matrices). In some embodiments, evaporating the solvent forms separate islands of the one or more polymer matrices.

In some embodiments, evaporating the solvent includes heating the solvent (or heating the plurality of food items 410 with the cannabinoid solution 420 applied thereon). In some embodiments, heating the solvent includes exposing the cannabinoid solution 420 on the plurality of food items 410 to heated gas (e.g., heated nitrogen above a boiling point of the solvent, such as 78.37° C. for ethanol, however, a slower evaporation process at a lower temperature may also be used). In some embodiments, evaporating the solvent includes exposing the solvent to a particular combination of temperature and pressure.

FIG. 5D illustrates that the plurality of food items with one or more polymer matrices thereon (e.g., food items coated with the polymer matrices) is mixed with a plurality of food items without one or more polymer matrices thereon. This forms a mixture of food items including (i) a first set of a plurality of food items with one or more polymer matrices remaining thereon; and (ii) a second set of a plurality of food items without the one or more polymer matrices remaining thereon.

In some cases, mixing one or more polymer matrices (e.g., not coated on food items) with a plurality of food items may lead to separation of (and aggregation of) the one or more polymer matrices from the plurality of food items over time (e.g., due to the size, density, and weight differences). Thus, a non-homogeneous mixture of the one or more polymer matrices and the plurality of food items may result from directly mixing the one or more polymer matrices with the plurality of food items. Instead of mixing the one or more polymer matrices directly with the plurality of food items, mixing a plurality of food items with one or more polymer matrices thereon and a plurality of food items without one or more polymer matrices thereon (as shown in FIG. 5D) reduces separation of the one or more polymer matrices from the plurality of food items.

In addition, mixing a plurality of food items with one or more polymer matrices thereon and a plurality of food items without one or more polymer matrices thereon facilitates adjusting an amount of cannabinoid compounds included in a container. An amount of the one or more cannabinoid compounds remaining on the plurality of food items after evaporation of the solvent depends on multiple factors (e.g., concentrations of the one or more cannabinoid compounds in the cannabinoid solution, an amount of the plurality of food items, a surface area of the plurality of food items, an affinity between surfaces of the plurality of food items and the one or more polymer matrices, etc.). Instead of adjusting the amount of the one or more cannabinoid compounds remaining on the plurality of food items, an amount of cannabinoid compounds included in a container can be adjusted (without changing the amount of the plurality of food items) by mixing the plurality of food items with one or more polymer matrices thereon and the plurality of food items without one or more polymer matrices thereon.

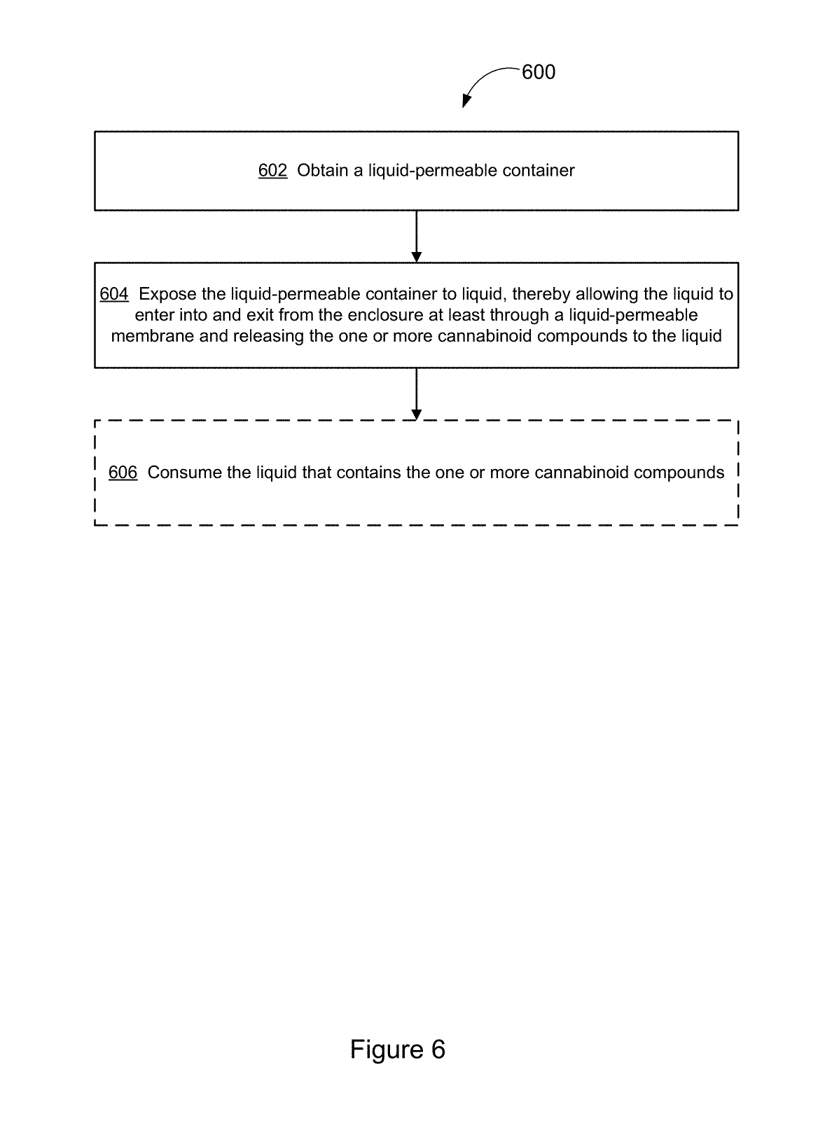

FIG. 6 illustrates a method 600 for preparing a beverage containing cannabinoids in accordance with some embodiments.

The method 600 includes obtaining (602) a liquid-permeable container (e.g., a liquid-permeable container described above with respect to FIGS. 2A-2D). In some embodiments, the liquid-permeable container includes an enclosure that includes a liquid-permeable membrane. The liquid-permeable membrane permits liquid to enter into and exit from the enclosure at least through the liquid-permeable membrane. The liquid-permeable container also includes one or more polymer matrices, that include one or more cannabinoid compounds.

The method 600 includes exposing (604) the liquid-permeable container to the liquid, thereby allowing the liquid to enter into and exit from the enclosure at least through the liquid-permeable membrane and releasing the one or more cannabinoid compounds to the liquid.

In some embodiments, the liquid is water. In some embodiments, the liquid is hot water. In some embodiments, the liquid is cold water.

In some embodiments, the method 600 includes consuming (606) the liquid that contains the one or more cannabinoid compounds.

In some embodiments, the liquid-permeable container is exposed to the liquid within a predefined time before consumption of the one or more cannabinoid compounds. In some embodiments, the predefined time is one hour. In some embodiments, the predefined time is one of: one day, twelve hours, six hours, three hours, two hours, one hour, thirty minutes, and fifteen minutes. In some embodiments, the predefined time is one of: one day, twelve hours, six hours, three hours, two hours, one hour, thirty minutes, fifteen minutes, ten minutes, five minutes, and three minutes.

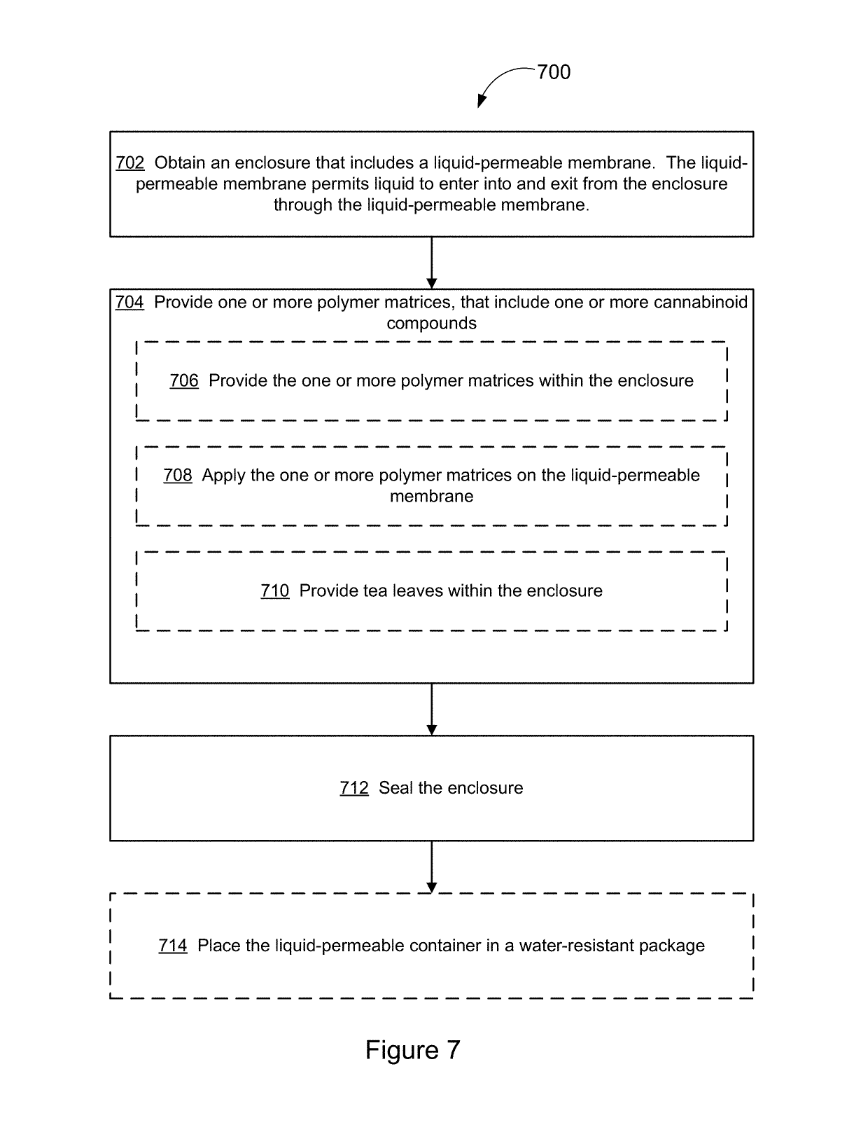

FIG. 7 illustrates a method 700 for manufacturing a liquid-permeable container in accordance with some embodiments.

The method 700 includes obtaining (702) an enclosure that includes a liquid-permeable membrane (e.g., an enclosure described above with respect to FIGS. 2A-2D). The liquid-permeable membrane permits liquid to enter into and exit from the enclosure through the liquid-permeable membrane.

The method 700 includes providing (704) one or more polymer matrices that include one or more cannabinoid compounds.

In some embodiments, the method 700 includes providing (706) the one or more polymer matrices within the enclosure.

In some embodiments, the method 700 includes applying (708) the one or more polymer matrices on the liquid-permeable membrane.

In some embodiments, the method 700 includes providing (710) tea leaves within the enclosure. In some embodiments, the method includes providing one or more flavoring substances within the enclosure.

The method 700 includes sealing (712) the enclosure.

In some embodiments, the method 700 includes placing (714) the liquid-permeable container in a water-resistant package (e.g., package 150 in FIG. 2D). In some embodiments, the water-resistant package includes plastic.

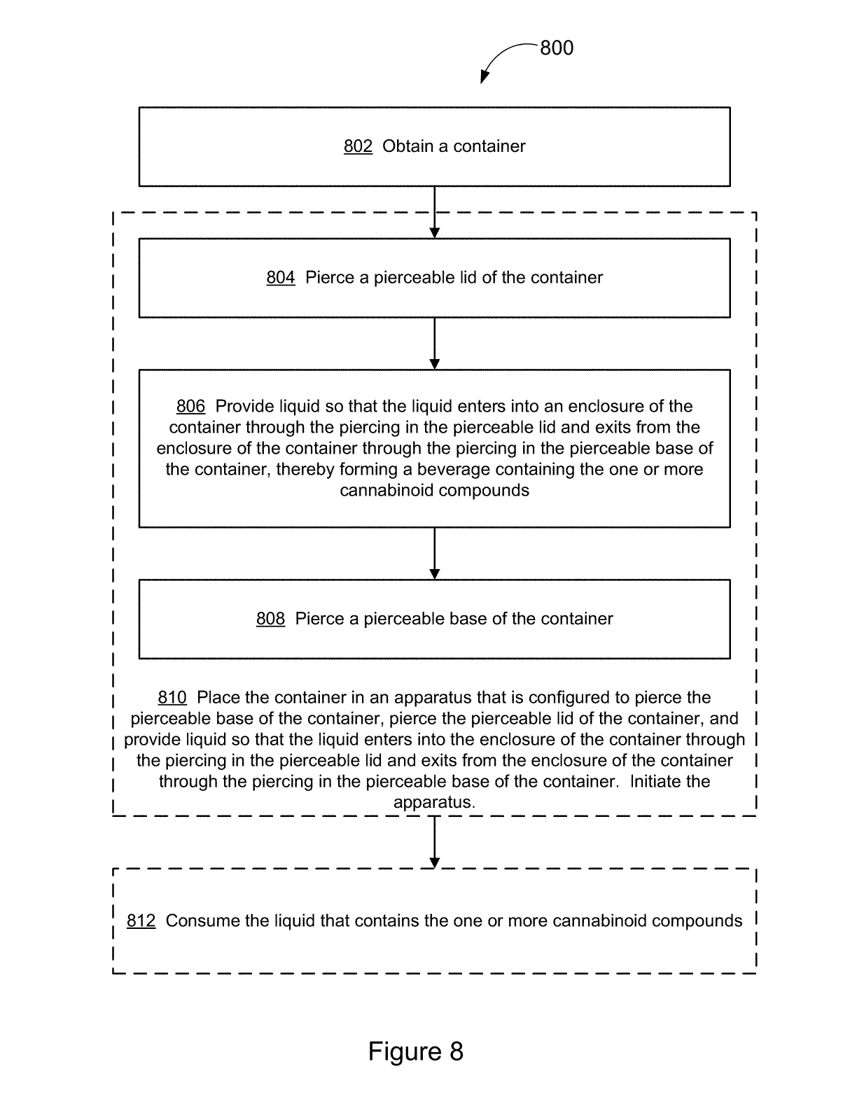

FIG. 8 illustrates a method 800 for preparing a beverage containing cannabinoids in accordance with some embodiments.

The method 800 includes obtaining (802) a container (e.g., a container described above with respect to FIG. 3). In some embodiments, the container for preparing a beverage includes an enclosure that includes one or more side walls, a pierceable base sealingly coupled with the one or more side walls, and a pierceable lid sealingly coupled with the one or more side walls opposite to the base; and one or more polymer matrices, that include one or more cannabinoid compounds, located within the enclosure.

The method 800 includes piercing (804) the pierceable lid of the container.

The method 800 includes providing (806) liquid so that the liquid enters into the enclosure of the container through the piercing in the pierceable lid and exits from the enclosure of the container through the piercing in the pierceable base of the container, thereby forming a beverage containing the one or more cannabinoid compounds. In some embodiments, the pierceable lid is pierced before providing the liquid. In some embodiments, the pierceable base is pierced after providing the liquid.

The method 800 includes piercing (808) the pierceable base of the container. In some embodiments, the pierceable lid is pierced before piercing the pierceable base. In some embodiments, the pierceable base is pierced before piercing the pierceable lid. In some embodiments, the pierceable lid and the pierceable base are pierced concurrently.

In some embodiments, the method 800 includes placing (810) the container in an apparatus (e.g., a single-serve beverage apparatus) that is configured to pierce the pierceable base of the container, pierce the pierceable lid of the container, and provide liquid so that the liquid enters into the enclosure of the container through the piercing in the pierceable lid and exits from the enclosure of the container through the piercing in the pierceable base of the container; and initiating the apparatus.

In some embodiments, the method 800 includes consuming (812) the beverage that contains the one or more cannabinoid compounds.

In some embodiments, the one or more cannabinoid compounds are introduced to the liquid within a predefined time before consumption of the one or more cannabinoid compounds. In some embodiments, the predefined time is one hour. In some embodiments, the predefined time is one of: one day, twelve hours, six hours, three hours, two hours, one hour, thirty minutes, and fifteen minutes.

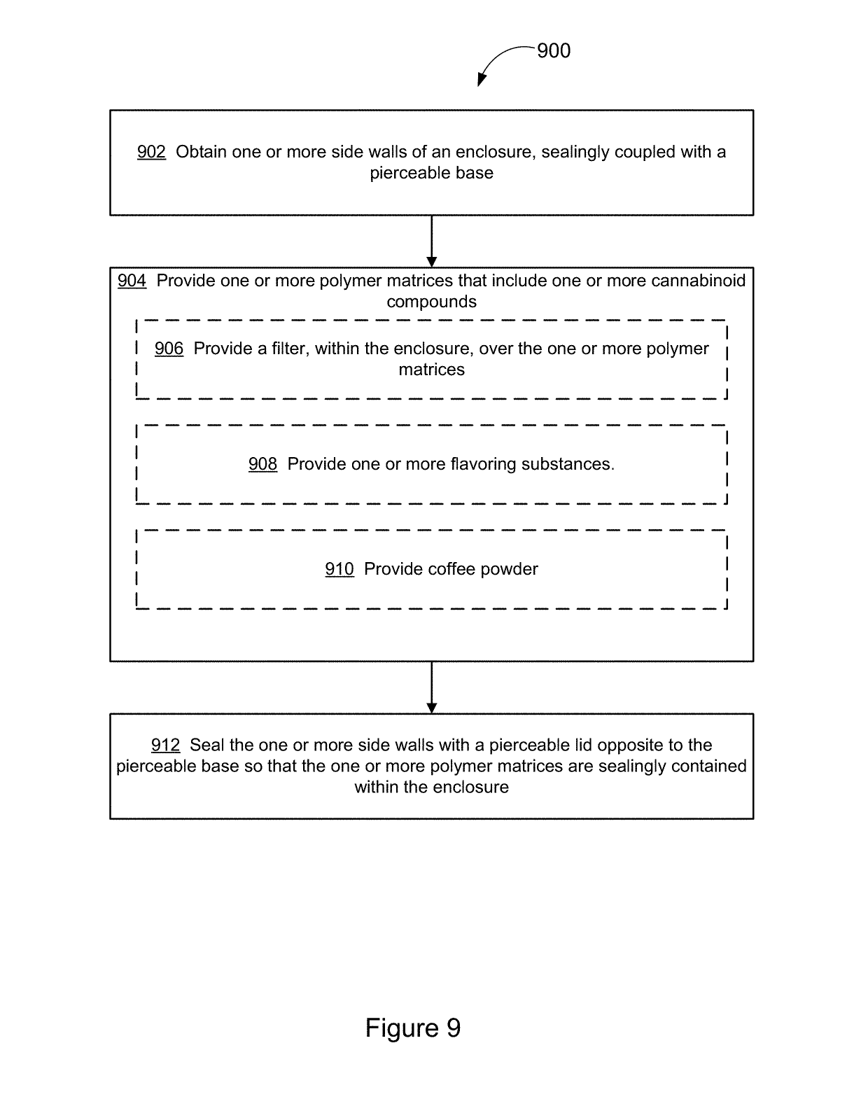

FIG. 9 illustrates a method 900 for manufacturing a container in accordance with some embodiments.

The method 900 includes obtaining (902) one or more side walls of an enclosure, sealingly coupled with a pierceable base (e.g., one or more side walls 210 and a pierceable base 220 described above with respect to FIG. 3). In some embodiments, the one or more side walls of the enclosure are integrally formed with the pierceable base. In some embodiments, the one or more side walls of the enclosure are attached to the pierceable base.

The method 900 includes providing (904) one or more polymer matrices that include one or more cannabinoid compounds.

In some embodiments, the method 900 includes providing (906) a filter, within the enclosure, over the one or more polymer matrices. In some embodiments, the method 906 includes providing the one or more polymer matrices after placing the filter within the one or more side walls of the enclosure.

In some embodiments, the method 900 includes providing (908) one or more flavoring substances.

In some embodiments, the method 900 includes providing (910) coffee powder. In some embodiments, the container includes cocoa powder. In some embodiments, the container includes soda extracts.

The method 900 includes sealing (912) the one or more side walls with the pierceable lid opposite to the pierceable base so that the one or more polymer matrices are sealingly contained within the enclosure.

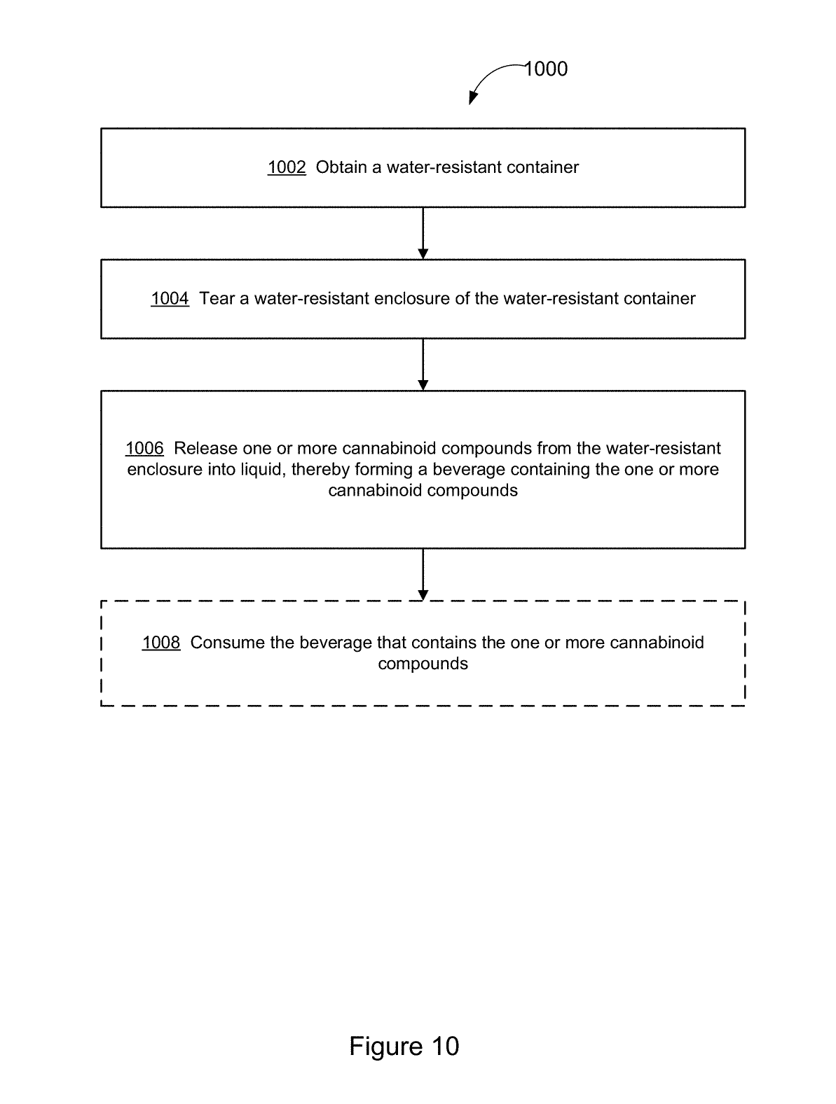

FIG. 10 illustrates a method 1000 for preparing a beverage containing cannabinoids in accordance with some embodiments.

The method 1000 includes (1002) obtaining a water-resistant container (e.g., a water-resistant container described above with respect to FIGS. 4A-4B). In some embodiments, the water-resistant container includes one or more polymer matrices that include one or more cannabinoid compounds; and a water-resistant enclosure enclosing the one or more polymer matrices.

The method 1000 includes (1004) tearing (e.g., opening, cutting, punching, or removing a portion of) a water-resistant enclosure of the water-resistant container.

The method 1000 includes (1006) releasing one or more cannabinoid compounds from the water-resistant enclosure into liquid, thereby forming a beverage containing the one or more cannabinoid compounds.

In some embodiments, the method 1000 includes (1008) consuming the beverage that contains the cannabinoid compounds.

In some embodiments, the one or more cannabinoid compounds are introduced to the liquid within a predefined time before consumption of the one or more cannabinoid compounds. In some embodiments, the predefined time is one hour. In some embodiments, the predefined time is one of: one day, twelve hours, six hours, three hours, two hours, one hour, thirty minutes, and fifteen minutes.

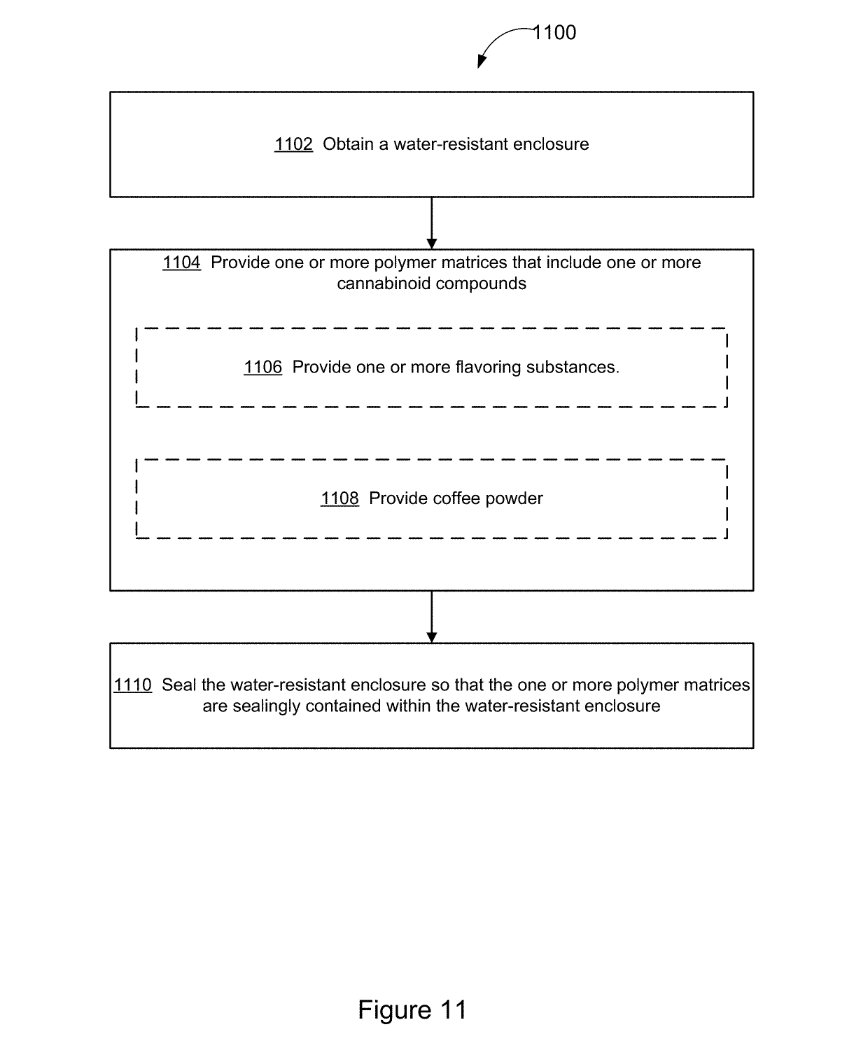

FIG. 11 illustrates a method 1100 for manufacturing a water-resistant container in accordance with some embodiments.

The method 1100 includes obtaining (1102) the water-resistant enclosure.

The method 1100 includes providing (1104) one or more polymer matrices that include one or more cannabinoid compounds.

In some embodiments, the method 1100 includes providing (1106) one or more flavoring substances.

In some embodiments, the method 1100 includes providing (1108) coffee powder. In some embodiments, the container includes cocoa powder. In some embodiments, the container includes a mixture of cocoa powder, sugar, and milk powder. In some embodiments, the container includes soda extracts.

The method 1100 includes sealing (1110) the water-resistant enclosure so that the one or more polymer matrices are sealingly contained within the water-resistant enclosure.

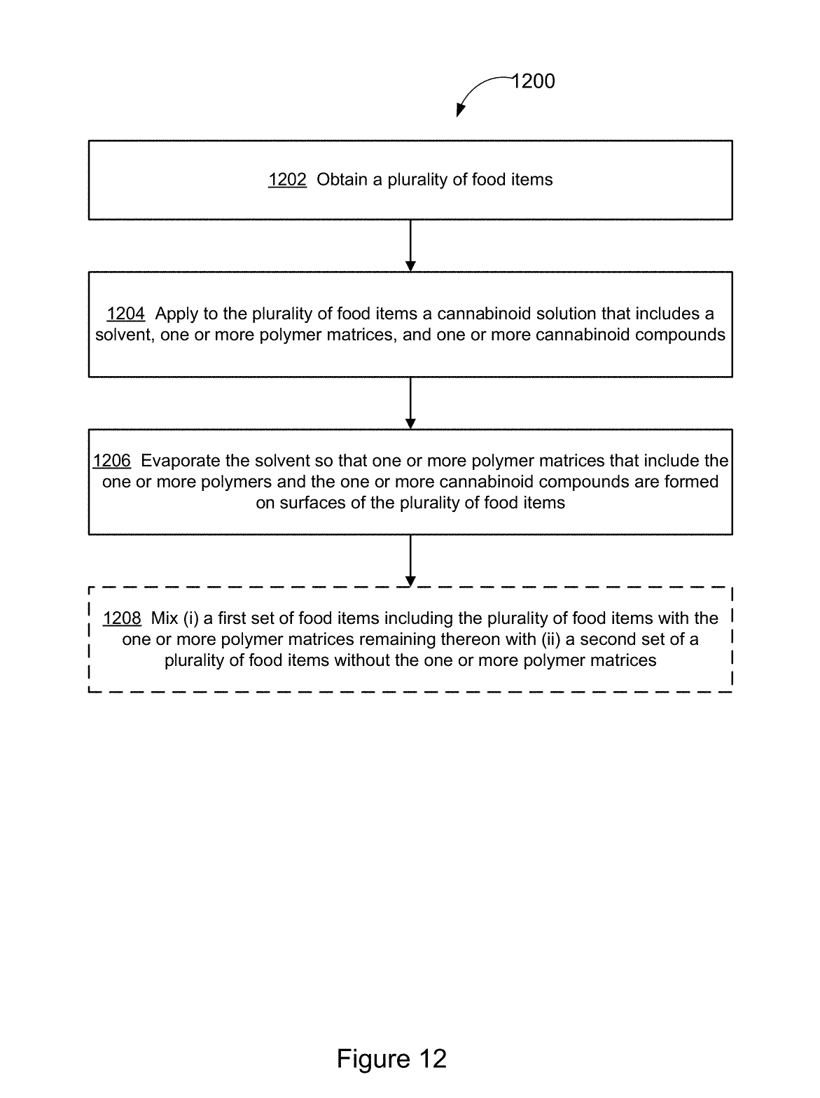

FIG. 12 illustrates a method 1200 for preparing a food item in accordance with some embodiments.

The method 1200 includes obtaining (1202) a plurality of food items (fruit, vegetables, meat, candy, baked goods, spices, etc.).

The method 1200 includes applying (1204) to the plurality of food items a cannabinoid solution that includes a solvent, one or more polymers, and one or more cannabinoid compounds. In some embodiments, the one or more polymers and the one or more cannabinoid compounds are dissolved in the solvent. In some embodiments, applying to the plurality of food items the cannabinoid solution includes separately applying a solvent, one or more polymers and one or more cannabinoid compounds. For example, the solvent may be applied first, followed by application of the one or more polymers, which is in turn followed by application of the one or more cannabinoid compounds. The sequence of adding the solvent, the one or more polymers, and the one or more cannabinoid compounds may vary.

In some embodiments, the one or more polymers include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide). In some embodiments, the one or more polymers are selected from a group consisting of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, the one or more cannabinoid compounds include one or more of: tetrahydrocannabinolic acid, tetrahydrocannabinol, and cannabidiol. In some embodiments, the one or more cannabinoid compounds include tetrahydrocannabinol. In some embodiments, the one or more cannabinoid compounds include cannabidiol. In some embodiments, the one or more cannabinoid compounds include tetrahydrocannabinolic acid.

In some embodiments, the solvent includes an organic solvent (e.g., ethanol, isopropanol, and dichloromethane).

In some embodiments, the cannabinoid solution includes food coloring. In some cases, the one or more polymer matrices that include the one or more cannabinoid compounds are odorless, colorless, and/or tasteless. To reduce the chance of a user unknowingly consuming a food item with one or more polymer matrices thereon, the food coloring can be used to indicate the presence of the one or more polymer matrices.

In some embodiments, applying the cannabinoid solution to the plurality of food items includes dousing the plurality of food items into or with the cannabinoid solution (e.g., soaking the plurality of food items into the cannabinoid solution, at least partially dipping the plurality of food items into the cannabinoid solution, or pouring the cannabinoid solution over the plurality of food items). In some embodiments, the cannabinoid solution is applied to entire surfaces of the plurality of food items. In some embodiments, the cannabinoid solution is applied to a portion of the surfaces of the plurality of food items.

In some embodiments, applying the cannabinoid solution to the plurality of food items includes atomizing the cannabinoid solution (e.g., spraying the cannabinoid solution) into multiple droplets and applying the multiple droplets of the cannabinoid solution to the plurality of food items.

The method 1200 includes evaporating (1206) the solvent so that one or more polymer matrices that include the one or more polymers and the one or more cannabinoid compounds are formed on surfaces of the plurality of food items. In some embodiments, in the one or more polymer matrices, the one or more polymers are chemically coupled with the one or more cannabinoid compounds. In some embodiments, the one or more polymer matrices, the one or more polymers are mechanically coupled with the one or more cannabinoid compounds.

In some embodiments, the one or more polymer matrices include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, evaporating the solvent includes heating the solvent. In some embodiments, evaporating the solvent includes exposing the solvent to a particular combination of temperature and pressure. In some embodiments, heating the solvent includes exposing the solvent to heated gas. In some embodiments, the heated gas is nitrogen.

In some embodiments, the method 1200 includes (1208) mixing (i) a first set of food items including the plurality of food items with the one or more polymer matrices remaining thereon with (ii) a second set of a plurality of food items without the one or more polymer matrices.

In some embodiments, the plurality of food items, in the first set of food items, with the one or more polymer matrices remaining thereon and the plurality of food items, in the second set of food items, without the one or more polymer matrices, include food items of a same type. For example, coffee grinds with one or more polymer matrices thereon and coffee grinds without one or more polymer matrices thereon are mixed.

In some embodiments, the plurality of food items includes coffee powder. In some embodiments, the plurality of food items includes coffee beans. In some embodiments, the plurality of food items includes tea leaves. In some embodiments, the plurality of food items includes tea balls. In some embodiments, the plurality of food items includes spices (e.g., salt, sugar, pepper, etc.).

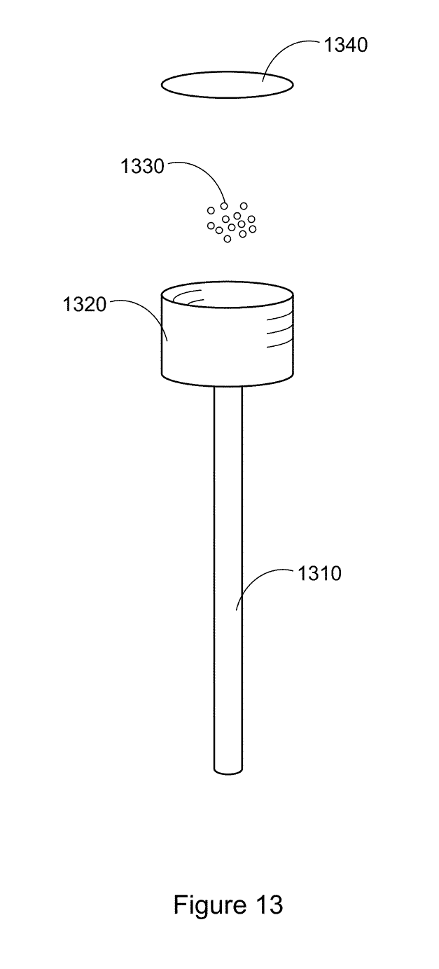

FIG. 13 illustrates a beverage stirrer in accordance with some embodiments.

The beverage stirrer includes rod 1310 and container 1320 coupled to a first end of rod 1310. Container 1320 releasably stores one or more cannabinoid compounds 1330. In some embodiments, container 1320 releasably stores one or more polymer matrices that include one or more cannabinoid compounds 1330. For example, container 1320 stores one or more polymer matrices until one or more release conditions are satisfied (e.g., liquid enters into container 1320 and mixes with one or more cannabinoid compounds 1330, and a mixture of the liquid and one or more cannabinoid compounds 1330 to exit from container 1320).

In some embodiments, container 1320 sealingly stores one or more cannabinoid compounds 1330. For example, container 1320 prevents release of one or more cannabinoid compounds 1330 until a certain condition is met (e.g., liquid enters container 1320 and/or membrane 1340 is opened).

In some embodiments, the one or more polymer matrices include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

In some embodiments, container 1320 includes membrane 1340.

In some embodiments, membrane 1340 includes a liquid-permeable membrane (e.g., membrane 1340 is a liquid-permeable membrane). The liquid-permeable membrane permits liquid to enter into and exit from the container at least through the liquid-permeable membrane.

In some embodiments, the beverage stirrer is configured to retain one or more cannabinoid compounds 1330 prior to container 1320 being exposed to liquid.

In some embodiments, the liquid is water.

In some embodiments, membrane 1340 includes a water soluble membrane (e.g., membrane 1340 is a water soluble membrane). The water soluble membrane is configured to prevent egress of one or more cannabinoid compounds 1330 until at least a portion of the water soluble membrane is dissolved.

In some embodiments, one or more cannabinoid compounds 1330 are applied on an inner surface of container 1320. In some embodiments, one or more polymer matrices that include one or more candidate compounds 1330 are applied on the inner surface of container 1320.

In some embodiments, one or more cannabinoid compounds 1330 are in a powder form. In some embodiments, at least a portion of one or more cannabinoid compounds 1330 is in a powder form (e.g., the one or more polymer matrices that include one or more cannabinoid compounds 1330 are in a powder form). Alternatively, or additionally, at least a portion of one or more cannabinoid compounds 1330 has a shape of a film (e.g., the one or more polymer matrices that include one or more cannabinoid compounds 1330 has a shape of a film).

In some embodiments, one or more cannabinoid compounds 1330 include one or more of: tetrahydrocannabinolic acid, tetrahydrocannabinol, and cannabidiol. In some embodiments, one or more cannabinoid compounds 1330 include tetrahydrocannabinol. In some embodiments, one or more cannabinoid compounds 1330 include cannabidiol. In some embodiments, one or more cannabinoid compounds 1330 include tetrahydrocannabinolic acid.

In some embodiments, the beverage stirrer includes one or more flavoring substances. In some embodiments, container 1320 includes the one or more flavoring substances.

In some embodiments, a potency of the one or more cannabinoid compounds 1330 decreases over time subsequent to the one or more cannabinoid compounds are exposed to the liquid.

In some embodiments, container 1320 includes any of the liquid-permeable containers described herein.

In some embodiments, membrane 1340 is a removable enclosure. For example, membrane 1340 is configured to be peeled off. A user, prior to using the beverage stirrer, peels off membrane 1340 and releases one or more polymer matrices in liquid. Alternatively, the removable enclosure has a shape other than a membrane (e.g., a cap, lid, stopper, etc.).

In accordance with some embodiments, a method for preparing a beverage containing cannabinoids includes obtaining a beverage stirrer with a container that releasably stores one or more cannabinoid compounds; and exposing the beverage stirrer to liquid, thereby releasing the one or more cannabinoid compounds to the liquid.

In some embodiments, the method includes stirring the liquid with the beverage stirrer.

In some embodiments, the method includes consuming the liquid that contains the one or more cannabinoid compounds.

In some embodiments, the liquid is water. In some embodiments, the liquid is hot water. In some embodiments, the liquid is cold water.

In some embodiments, the beverage stirrer is exposed to the liquid within a predefined time before consumption of the one or more cannabinoid compounds. In some embodiments, the predefined time is one hour. In some embodiments, the predefined time is one of: one day, twelve hours, six hours, three hours, two hours, one hour, thirty minutes, and fifteen minutes.

In accordance with some embodiments, a method includes obtaining a beverage stirrer that comprises a rod and a container coupled to a first end of the rod; placing in the container one or more polymer matrices that include one or more cannabinoid compounds; and sealing the container.

In some embodiments, sealing the container includes sealing the container with a liquid-permeable membrane. In some embodiments, the method includes applying the one or more polymer matrices on the liquid-permeable membrane.

In some embodiments, sealing the container includes sealing the container with a water-soluble membrane.

In some embodiments, the method includes placing the beverage stirrer in a water-resistant package.

In some embodiments, the water-resistant package includes plastic.

In accordance with some embodiments, a package includes a beverage stirrer and a water-resistant package enveloping the beverage stirrer.

While particular embodiments are described above, it will be understood that the scope of claims are not limited to these particular embodiments. On the contrary, the claims include alternatives, modifications and equivalent embodiments that are within the spirit and scope of the appended claims. Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

The terminology used in the description herein is for the purpose of describing particular embodiments only and is not intended to limit the scope of claims. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, operations, elements, and/or components, but do not preclude the presence or addition of one or more other features, operations, elements, components, and/or groups thereof.

It will also be understood that, although the terms first, second, etc. may be used herein to describe various elements, these elements should not be limited by these terms. These terms are only used to distinguish one element from another. For example, a first set of food items could be termed a second set of food items, and, similarly, a second set of food items could be termed a first set of food items, without departing from the scope of the claims. The first set of food items and the second set of food items are both sets of food items, but they are not the same set of food items.

The foregoing description, for purpose of explanation, has been described with reference to specific embodiments. However, the illustrative discussions above are not intended to be exhaustive or to limit the scope of the claims to the precise forms disclosed. Many modifications and variations are possible in view of the above teachings. The embodiments were chosen and described in order to best explain the principles described herein and their practical applications, to thereby enable others skilled in the art to best utilize various embodiments with various modifications as are suited to the particular use contemplated.

What is claimed is:

1. A beverage stirrer, comprising:
a rod; and
a container coupled to a first end of the rod, the container releasably storing one or more cannabinoid compounds.

2. The beverage stirrer of claim 1, wherein the container releasably stores one or more polymer matrices that include one or more cannabinoid compounds.

3. The beverage stirrer of claim 2, wherein the one or more polymer matrices include one or more of: polyethylene oxide, polyvinyl alcohol, hydroxypropyl methyl cellulose, polyoxazoline, polyethylene glycol, polyvinyl pyrrolidone, polyvinyl-pyrrolidone polyvynylacetate copolymer, hydroxypropyl cellulose, polyurethane, poloxamer, polyvinyl caprolactam-polyvinyl acetate-polyethylene glycol graft copolymer, pectin, chitosan, polycarbophil, alginate, hyaluronan, agar, dextran, pullulan, polyglycolide, and poly(DL-lactide-co-glycolide).

4. The beverage stirrer of claim 1, wherein the container includes a liquid-permeable membrane, wherein the liquid-permeable membrane permits liquid to enter into and exit from the container at least through the liquid-permeable membrane.

5. The beverage stirrer of claim 1, wherein the beverage stirrer is configured to retain the one or more cannabinoid compounds prior to the container being exposed to liquid.

6. The beverage stirrer of claim 1, wherein the container includes a water soluble membrane that is configured to prevent egress of the one or more cannabinoid compounds until at least a portion of the water soluble membrane is dissolved.

7. The beverage stirrer of claim 1, wherein the one or more cannabinoid compounds are located on an inner surface of the container.

8. The beverage stirrer of claim 1, wherein the one or more cannabinoid compounds are in a powder form.

9. The beverage stirrer of claim 1, wherein the one or more cannabinoid compounds include one or more of: tetrahydrocannabinolic acid, tetrahydrocannabinol, and cannabidiol.

10. The beverage stirrer of claim 1, further comprising one or more flavoring substances within the container.

11. The beverage stirrer of claim 1, wherein a potency of the one or more cannabinoid compounds decreases over time after the one or more cannabinoid compounds are exposed to the liquid.

12. A method for preparing a beverage containing cannabinoids, comprising:
obtaining the beverage stirrer of claim 1; and
exposing the beverage stirrer to liquid, thereby releasing the one or more cannabinoid compounds to the liquid.

13. The method of claim 12, further comprising:
stirring the liquid with the beverage stirrer.

14. The method of claim 12, wherein the beverage stirrer is exposed to the liquid within a predefined time before consumption of the one or more cannabinoid compounds.

15. A method of manufacturing the beverage stirrer of claim 1, comprising:
obtaining a beverage stirrer that comprises a rod and a container coupled to a first end of the rod;
placing in the container one or more cannabinoid compounds; and
sealing the container.

16. The method of claim 15, wherein sealing the container includes sealing the container with a liquid-permeable membrane.

17. The method of claim 15, including applying the one or more cannabinoid compounds on the liquid-permeable membrane.

18. The method of claim 15, wherein sealing the container includes sealing the container with a water-soluble membrane.

19. The method of claim 15, further comprising:
placing the beverage stirrer in a water-resistant package.

20. The method of claim 19, wherein the water-resistant package includes plastic.
